(19) United States
(12) Patent Application Publication
(10) Pub. No.: US 2007/0048252 A1
(43) Pub. Date: Mar. 1, 2007

(54) Conjugates of the hydrophilic polymer and the molecules from boxwood extraction, and pharmaceutical compositions of the conjugates

(75) Inventors: Dequan Zhu, Beijing (CN); Shishan Ji, Beijing (CN)

Correspondence Address: GLOBAL IP SERVICES, 2462 ROCK ST., APT. 6, MOUNTAIN VIEW, CA, 94043, US

(73) Assignee: Beijingn Jenkem Technology CO., Ltd., Haidian District, Beijing (CN)

(21) Appl. No.: 10/595,718

(22) PCT Filed: Nov. 5, 2004
(86) PCT No.: PCT/CN04/01259

§ 371 Date: May 31, 2006

(30) Foreign Application Priority Data
Nov. 7, 2003 (CN) CN200310103179.5

(51) Int. Cl. A61K 31/765; C07J 53/00; A61K 31/56

(52) U.S. Cl. 424/78.38; 514/169; 552/512

(57) ABSTRACT

The present invention relates to a group of conjugates of the hydrophilic polymers and the molecules from boxwood extraction. PEGylation process or the process alike was used to generate the conjugates, which have increased water solubility and prolonged the circulation half-life in the body.

### FIELDS OF THE INVENTION

The present invention relates to a group of conjugates of the hydrophilic polymers and the molecules from boxwood extraction, and pharmaceutical compositions consisting the conjugates, and the pharmaceutical usage of the conjugates too.

### BACKGROUND OF THE INVENTION

The molecules from boxwood extraction are commonly referred as to Buxus alksloid, including Cyclovirobuxine D (CVB), Cycloprotobuxine A, Cycloprotobuxine C, Cyclovirobuxine C, et al.

Boxwood is a type of Buxus microphylla Sieb, a plant of Buxus family, which grows in mide-east of China. As a Chinese traditional medicine, it has been widely used in treating cardiovascular diseases. The active ingredient, Cyclovirobuxine D (CVB) collected after extraction and purification, has been formulated into tablets, which show very satisfactory effect in treatment of coronary heart diseases.

Cyclovirobuxine D has many therapeutic potentials, such as reducing oxygen consumption of the heart muscle, increase the strength of the heart, improve the blood flow in coronary arteries, prevent arrhythmia, reduce coronary heart attach. However, Cyclovirobuxine D has very low water solubility. Currently, it can only formulated as tablet for oral consumption. Injectables have yet been available. Although Cyclovirobuxine D can be solubilized by adding acidic substance, it will precipitate eventually when injected into the body, causing adverse effect such as toxicity and so on. In addition, Cyclovirobuxine D has short circulation half-life, so patients need to take many dose per day to ensure the therapeutic effects. In order to increase the circulation half-life, stability, targeting effect, and the bioavailability, present invention provides a solution for the molecules by conjugation with water hydrophilic polymers.

Currently, PEG derivatives have been widely used to conjugate proteins, peptides or other therapeutic molecules to prolong the physiological half-life, to lower the immunogenicity and toxicity. In clinical applications, PEGs and its derivatives have been often used as carriers in commercial drug formulations. The attempt of conjugating PEG to drug molecule has made an impressive progress during last 10 years and had been successfully applied to many drugs approved on the market. For example, PEG-intron®, a conjugate of PEG and a type of α-interferon, exhibits longer circulation half-life and better therapeutic effect. The conjugate of PEG and paclitaxel shows reduced toxicity and increased bioactivity. The metabolism procedure of PEG is also well known that makes PEG a safe drug modifier.

PEGylation is often referred as to a process in which one or two of the terminal groups of the PEG are activated to form a proper functional group, and then is reacted with at least one functional group of the drug to form a stable linkage.

The object of present invention is to generate a group of conjugates of hydrophilic polymers and the molecules from boxwood extraction by a PEGylation process or another process alike. The resulting conjugates have improved water solubility and prolonged circulation half time, which ensure appropriate drug concentration in blood and possibly sustained release effect.

### SUMMARY OF THE INVENTION

The present invention relates to a group of conjugates of hydrophilic polymers and the molecules obtained from boxwood extraction. Wherein, said molecules include but not limited by Cyclovirobuxine D, Cycloprotobuxine A, Cycloprotobuxine C, Cyclovirobuxine C et al; said hydrophilic polymers are polyethylene glycol, polyglutamic acid, polyaspartatic acid, polypropylene, polyvinyl alcohol, polyacrylmorpholine and their copolymer thereof.

In one aspect of the invention, the present invention is to provide a conjugate of hydrophilic polymers and the molecules from boxwood extraction or the synthetic derivatives of the molecules, represented by formula I:
(P-L [symbol] ₙD (I)
wherein:

P is a hydrophilic polymer, which is selected from a group consisting of polyethylene glycol, polyglutamic acid, polyaspartatic acid, polypropylene, polyvinyl alcohol, polyacrylmorpholine and their copolymer thereof;

n is an integer, not exceed the total number of hydroxy and amine group on the D;

L is a linking group, which is selected from a group consisting of carboxylic acid, ester, carbonate, urethane, ether, acetal, and amide;

D is a molecule from the extraction of boxwood or its synthetic derivative, selected from a group consisting of Cyclovirobuxine D, Cycloprotobuxine A, Cycloprotobuxine C, Cyclovirobuxine C and their derivatives;

Preferably, the present invention is to provide a conjugate of hydrophilic polymers and the molecule from extraction of boxwood by formula (I-D₁): 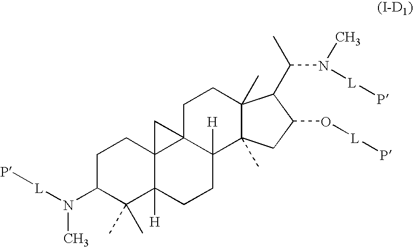
wherein:

P′ is independently selected from H or P, but not all be H at the same time; and

L is a linker group as disclosed previously.

In another aspect of the invention, the present invention is to provide a conjugate of hydrophilic polymers multicarboxyl oligopeptide and the molecule from extraction of boxwood or the synthetic derivatives of the molecules, represented by formula (II): 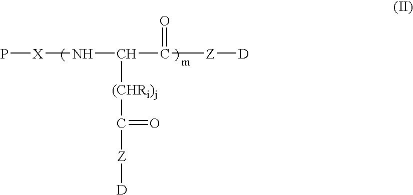
wherein:

P is a hydrophilic polymer, which is selected from a group consisting of polyethylene glycol, polyglutamic acid, polyaspartatic acid, polypropylene, polyvinyl alcohol, polyacrylmorpholine and their copolymer thereof;

m is an integer from 2 to 12;

j is an integer from 1 to 6;

Rᵢ is a group selected from a group consisting of H, C₁₋₁₂ alkyls, substituted aryls, aralkyls, heteroalkyls and substituted alkyls;

X is a linking group, selected from (CH₂)ₖ, (CH₂)ₖOCO, (CH₂)ₖNHCO or (CH₂)ₖCO, and k is 0-10;

Z is a linking group selected from O, NH, NHR, O(CH₂)ₕCOO or NH(CHR)ₕCOO, and h is 1-10;

D is the molecule from extraction of boxwood or its derivative, preferring Cyclovirobuxine D.

In another aspect of the invention, the present invention is to provide pharmaceutical composition comprising the conjugate as active ingredient.

In another aspect of the invention, present invention provide a method to prolong therapeutic half-life of the drug in vivo through conjugation of the active drug molecule to the hydrophilic polymer. Also, the hydrophilic polymer can provide protection for the drugs conjugated to the polymer, improving the drugs' stability and hydrophilic, prolonging the therapeutic life in vivo, and improving bioavailability.

### DETAILED DESCRIPTION OF THE INVENTION

The bioactive molecules from extraction of boxwood are Buxus alksloid. They are obtained mainly from the Buxus microphylla Sieb. et. The primary component is Cyclovirobuxine D, which has following structure (D₁):

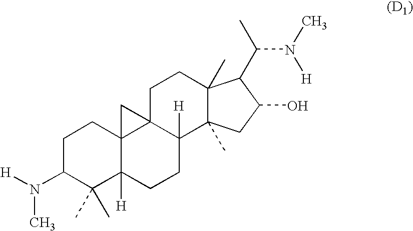

The conjugates of the invention were prepared by following method: modified the end group of hydrophilic polymer to get the appropriate active group, and then conjugated the polymer to the amine or hydroxyl group of the Buxus alksloid molecule, such as Cyclovirobuxine D, Cycloprotobuxine A, Cycloprotobuxine C, Cyclovirobuxine C. A conjugation method was also obtained to selectively conjugate the polymer to the hydroxyl group in the alkaloid molecule to preserve the alkaloid properties.

From now on, polyethylene glycol (PEG) is used as the example of the hydrophilic polymer for illustration. But it should be understood that the hydrophilic polymer in present invention should include polyethylene glycol's copolymer and other water-solubility polymers, such as polyglutamic acid, polyaspartatic acid, polypropylene, polyvinyl alcohol, polyacrylmorpholine and their copolymer thereof.

The general structure of polyethylene glycol (PEG) is as the formula below: 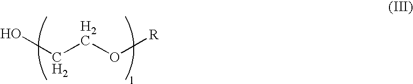
wherein:

R is H or C₁₋₁₂ alkyls;

n is an integer, representing the degree of the polymerization;

For R being a lower alkyl, R can be of any lower alkyl groups having 1-6 carbon atoms, for examples, methyl, ethyl, n-propyl, iso-propyl, n-butyl, iso-butyl, n-amyl, iso-amyl, and n-hexyl. For R is a cycloalkyl, a cycloalkyl consists of 3-7 carbon atoms is preferred, for example, cyclopropyl, cyclobutyl, and cyclohexyl. Among those, cyclohexyl is preferred choice. The typical compound is methoxy-polyethylene glycol (mPEG). Other analogs and derivatives of polyethylene glycol can also apply to the present invention, for example, polypropylene, polyol, and polyacrylmorpholine etc.

In respect of PEG, it is usually measured by molecular weight. It's preferred the molecular weight of PEG which forms the conjugate falls in the range from 300 to 60000 Dalton, which means n about 6˜1300. It's more preferred that n is 28, 112 or 450, and that means the molecular weight about 1325, 5000, and 20000 accordingly. Because of the heterogeneity of the PEG, it is usually incorrect to define PEG with self-repeating unit n. Therefore, PEG is normally characterized with average molecular weight. The starting PEG compounds with different molecular weight are readily synthesized using the known methods of the art or are available from commercial sources.

Certainly, besides of linear polymer, branched or other structure's can also be used in conjugation of the active molecules from the extraction of boxwood, such as Y-shaped branched and U-shaped branched PEG It's based on the drug molecule and the proposed property of the final conjugate to choose the suitable PEG structure.

If amino acids are used as the staring material, the activated polymer will have carboxylic groups. Especially, if the acidity amino acid or the polymer which consists of acidity amino acid are used, multi-carboxylic groups will be obtained. These structures will improve the loading of the small molecules from nature products, and will obtain sustained release by biodegradation in vivo.

In present invention, a group of oligopeptides containing multi-carboxylic groups was obtained from condensation of glutamic acid or aspartic acid. The said oligopeptide was then attached to the hydrophilic polymer to provide multiple carboxylic acid groups to the hydrophilic polymers. Such structure can conjugate to multiple hydrophobic drugs to one hydrophilic polymer. The resulting conjugates have increased water solubility, and significantly improved pharmacokinetics. More importantly, such multiple attachment can provide better sustained release of the active molecule, and hopefully provide better therapeutic efficacy.

In the present invention, the molecules from boxwood extraction were obtained from Chinese traditional medicine preparation. In such a way, four compounds are the major molecules: Cyclovirobuxine D, Cycloprotobuxine A, Cycloprotobuxine C and Cyclovirobuxine C. In addition to Cyclovirobuxine D, the structures of other molecules are as follows:

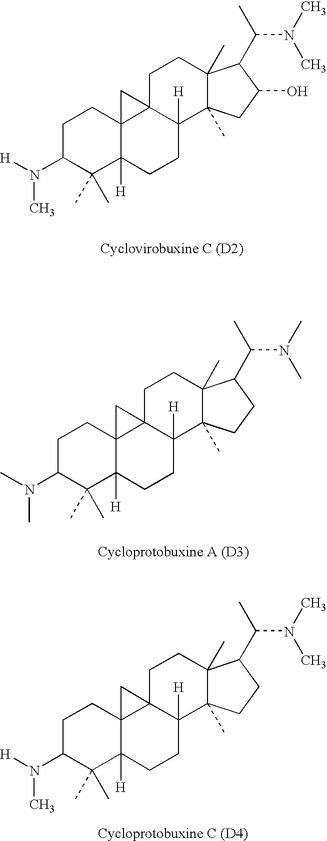

The molecules obtained from boxwood contain hydroxyl group and amine group. Conjugation can be achieved by attaching hydrophilic polymers to these function groups through ester, carbonate, amid or urethane linkages. Specially, the ester linkages can gradually release the molecule in the body by biodegradation. Currently, these molecules from boxwood extraction have been used in Chinese medicine market as oral tablets. But these molecules as oral drugs have low bioavailability, and must be taken several times a day. There is no perantal route for such molecules because poor water solubility and narrow therapeutic windows. In present invention, the conjugates of these molecules have good water solubility, fast action and longer circulation life time, suitable as injectable formulation for acute heart and brain disease.

The conjugate of the present invention are readily administered in the form of pure compound or suitable pharmaceutical composition, by the way of any acceptable methods or being included in the reagent of the similar usage. So, in another aspect of the invention, the present invention is to provide pharmaceutical composition comprising the conjugate as active ingredient.

Thus, the conjugate can be administered by oral, nasal, non-gastrointestinal, topical, transdermal, rectal and injection routes in the form of solid, semisolid, freeze dried powder or liquid dosage forms: for example, tablets, suppositories, pills, soft and hard gelatin capsules, powder, solution, suspention and aerosols. Preferably the unit dosage is suitable for a precise-dosage and easy administration. The composition includes general medicine carrier or excipient and the conjugate in the present invention as active ingredient (one or more). Furthermore, it also can include other reagent, carrier and excipient.

Generally speaking, depends on the way of administration, pharmaceutically acceptable composition will include about 1-99 wt. % the conjugate of the present invention, and 99-1 wt. % suitable pharmaceutical excipient. Preferably includes 5-75 wt. % the conjugate and the rest is suitable pharmaceutical excipient.

The preferable way of administration is injection, adopting general daily dosage scheme. The scheme is adjusted based on the situation of illness. The composition in present invention or pharmaceutically acceptable salts constitute the dosage for injection, for example, 0.5-50% active components dissolved in liquid pharmaceutical carrier, such as water, saline, aqueous glucose, glycerol, ethanol etc.

The compositions which are readily administered as liquid such as solutions and suspentions can be prepared by dissolving and dispersing the conjugate of the present invention (about 0.5-20%) and optionally the pharmaceutical excipient into carrier. The embodiment of carrier is water, saline, aqueous glucose, glycerol, ethanol etc.

If needed, the composition of the present invention can further include some adjuvant such as wetting agent, emulsifier, pH buffer, antioxidant etc. For example citric acid, anhydrous sorbital monolaurate, triethanolamine oleate, butylated hydroxytoluene etc., can be added.

The practical preparation methods of such dosage forms are known or obvious to the technician in the art, for example see Ramington's Pharmaceutical Sciences, 18^{th} edition, (Mack Publishing Company, Easton, Pa., 1990). In any case, according to the techniques of the present invention, the composition applied will include the effective amount of conjugate of the present invention for the treatment of corresponding disease.

### EXAMPLES

These examples do not intend to limit the scope of the invention by any means. The scope of the present invention is restricted by the Claims.

### Example 1

### Synthesis of the Conjugate of Poly(Ethylene Glycol) Acetic Acid and Cyclovirobuxine D Having Amide Linkage

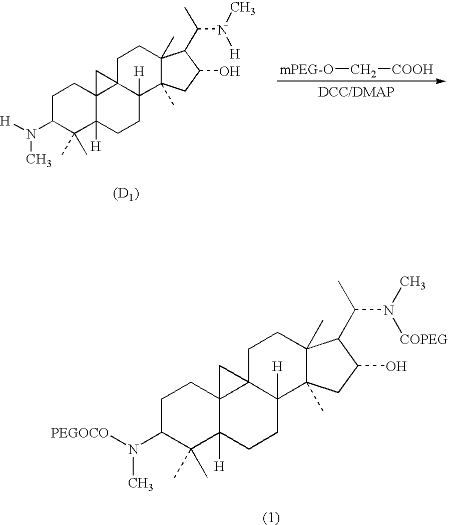

5.0 g α-methoxy polyethylene glycol acetic acid (mPEG-O—CH₂—COOH, Mw5000) 0.25 g Cyclovirobuxine D (D₁) and 0.2 g 4-dimethylamine pyridine (DMAP) were dissolved in 50 ml anhydrous dichloromethane. After that, 0.32 g dicyclohexylcarbodiimide (DCC) was added. Reaction mixture was stirred for a whole night at room temperature protected by nitrogen gas. The solvent was removed by rotary evaporation and the residue was dissolved in 20 ml of 1,4-dioxane. The undisolved solid was filtered off. The liquid was concentrated and precipitated in 100 ml of isopropyl alcohol. The product (N,N′-di(α-methoxy-ω-carboxyl-polyethylene glycol)-Cyclovirobuxine D (1)) was filtered and dried under vacuum. Yield: 4.2 g (83%). M.p. 57-59° C.

### Example 2

### Synthesis of the Conjugate of Poly(Ethylene Glycol) Derivative Containing Multicarboxylic Acid Dipeptide and Cyclovirobuxine D Having Amide Linkages

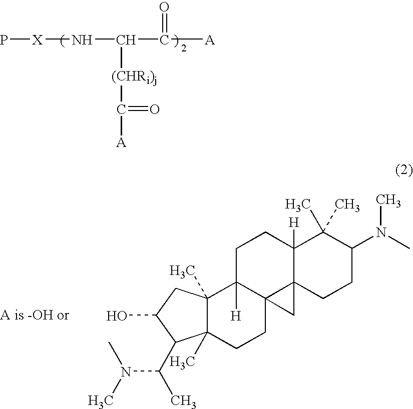

Preparation method was same as that in Example 1, while α-methoxy polyethylene glycol acetic acid was replaced with poly(ethylene glycol)-ω-glu-glu-dipeptide (Mw10500), and the product was N-(α-methoxy-polyethylene glycol-ω-glu-glu)-Cyclovirobuxine D (2). Yield: 90%. M.p. 58-59° C.

### Example 3

### Synthesis of the Conjugate of Polyglutamic-Acid and Cyclovirobuxine D Having Amide Linkages

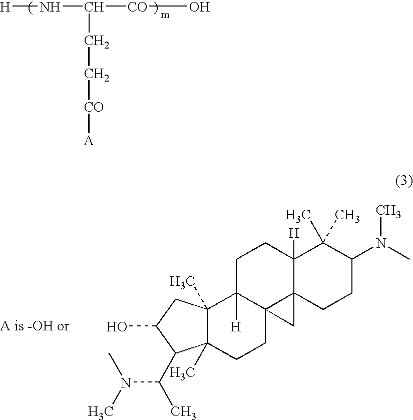

The preparation was the same as that in Example 1, while α-methoxy polyethylene glycol ethyl acid was replaced with polyglutamic-acid (Mw 5000), product is N-(polyglutamic-acid)-Cyclovirobuxine D (3). Yield: 90%.

### Example 4

### Synthesis of the Conjugate of Poly(Ethylene Glycol) Acetic Acid and Cyclovirobuxine D Having Ester Linkages

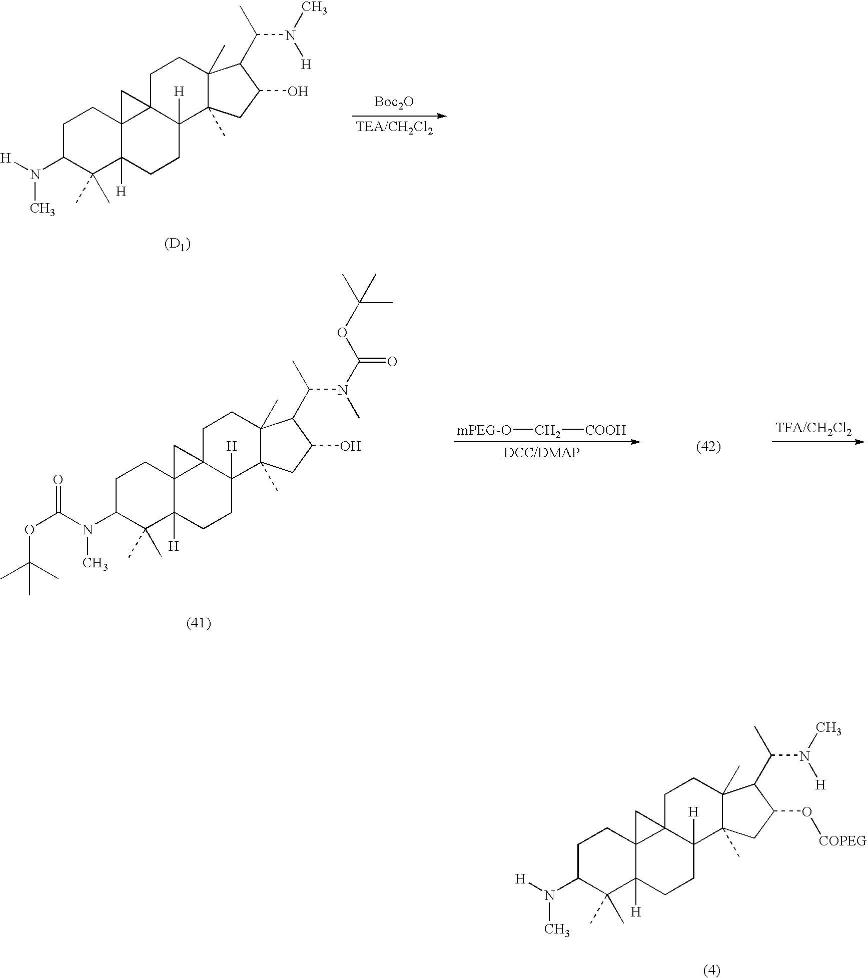 1.0 g of Cyclovirobuxine D and 0.8 ml triethylamine (TEA) were dissolved in 10 ml of anhydrous dichloromethane (CH₂Cl₂). 1.2 g di-tertiary-butyoxyl dicarbonate hydrohide in 10 ml anhydrous dichloromethane was added to the solution drop by drop in a period of 10 minute with ice-bath. Reaction mixture was stirred for 3 hr at room temperature. The solution was concentrated under reduced pressure. To it was added 20 ml of isopropyl alcohol. The solution was cooled down by ice bath. The crystal like solid was filtered, and washed with isopropyl alcohol twice. The product was collected and dried under vacuum. N,N′-di(tertiary-butyoxylcarboxyl)-Cyclovirobuxine D (41). Yield: 1.06 g, NMR (DMSO): 0.37(s,1H), 0.52(s,1H), 0.79(t,6H), 1.38(s,18H), 7.11(s,1H). Mp: 125-129° C.

0.5 g α-methoxy polyethylene glycol acetic acid (Mw10000) and 80 mg N,N′-di(tertiary-butyoxylcarboxyl)-Cyclovirobuxine D (41) (obtained in the previous step, Mw1250), 18 mg 4-dimethylamine pyridine (DMAP) were dissolved in 15 ml of anhydrous dichloromethane. 30 mg dicyclohexylcarbodiimide (DCC) were added thereafter. Reaction mixture was stirred over night at room temperature. Precipitate was removed by filtration. The filtrate was then concentrated and added into 5 ml of isopropyl alcohol and 30 ml of ethyl ether. The product (α-methoxy polyethylene glycol acetic acid-N,N′-di(tertiary-butyoxylcarboxyl)-Cyclovirobuxine D ester (42)) was collected by filtration, and dried under vacuum. Yield: 0.456 g, NMR (DMSO): 0.37(s,1H), 0.52(s,1H), 0.79(t,6H), 3.5(br m, PEG-H), 1.38(s,18H).

0.4 g α-methoxy-polyethylene-glycol-acetic-acid-N,N′-di(tertiary-butyoxylcarboxyl)-Cyclovirobuxine D ester (42) (obtained in the previous step) was dissolved in 5 ml of chloroform, then 1.5 ml trifluoroacetic acid (TFA) was added. Reaction mixture was stirred for 3 hours at room temperature, color change from colorless to slight green. The solution was concentrated under reduced pressure. The obtained mixture was added into 20 ml of ether. The precipitate (α-methoxy-polyethylene-glycol-acetic-acid-Cyclovirobuxine D ester (4)) was collected by filtration and dried under vacuum. Yield: 0.365 g, NMR (DMSO): 0.37(s,1H), 0.52(s,1H), 0.79(t,6H), 3.5(br m, PEG-H).

### Example 5

### Synthesis of the Conjugate of Poly(Ethylene Glycol) Derivative Containing Multicarboxylic Acid Tripeptide and Cyclovirobuxine D Having Ester Linkages

1.0 g α-methoxy-polyethylene-glycol-ω-glu-glu-glu (Mw10800) and 380 mg N,N′-di(tertiary-butyoxylcarboxyl)-Cyclovirobuxine D (41), 18 mg 4-dimethylamine pyridine (DMAP) were dissolved in 15 ml of anhydrous dichloromethane. 30 mg dicyclohexylcarbodiimide (DCC) were added thereafter. Reaction mixture was stirred over night at room temperature. Precipitate was removed by filtration. The filtrate was then concentrated and added into 5 ml of isopropyl alcohol and 30 ml of ethyl ether. The product (α-methoxy-polyethylene-glycol-glu-glu-glu-N,N′-di(tertiary-butyoxylcarboxyl)-Cyclovirobuxine D ester (51)) was collected by filtration and dried under vacuum. Yield: 0.456 g.

0.4 g α-methoxy-polyethylene-glycol-glu-glu-glu-N,N′-di(tertiary-butyoxylcarboxyl)-Cyclovirobuxine D ester (51) (obtained in the previous step) was dissolved in 5 ml chloroform, then 4 ml trifluoroacetic acid (TFA) was added. Reaction mixture was stirred for 3 hours at room temperature, color change from colorless to slight green. The solution was concentrated under reduced pressure. The obtained mixture was added into 20 ml of ether. The precipitate was collected by filtration and dried under vacuum. α-methoxy-polyethylene-glycol-glu-glu-glu-Cyclovirobuxine D ester (5). Yield: 0.365 g.

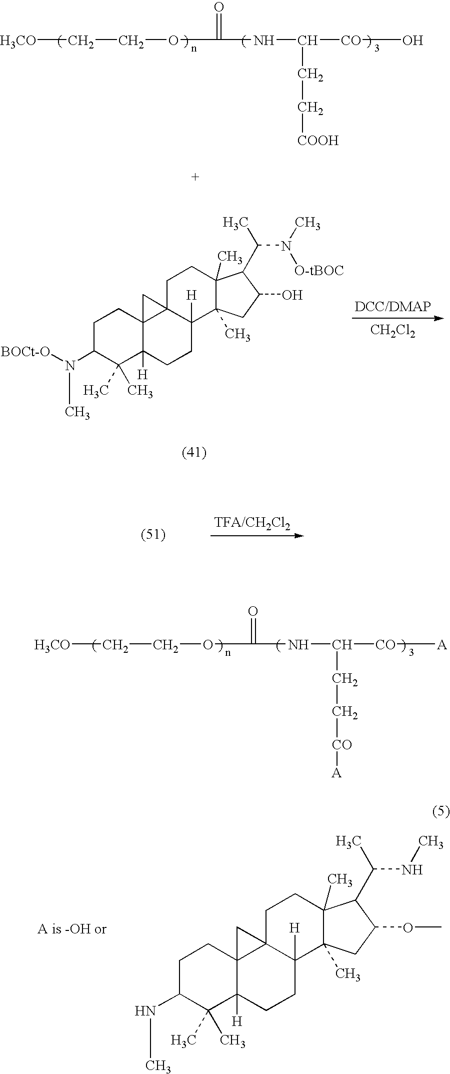

### Example 6

### Synthesis of the Conjugate of Polyglutamic-Acid and Cyclovirobuxine D Having Ester Linkages

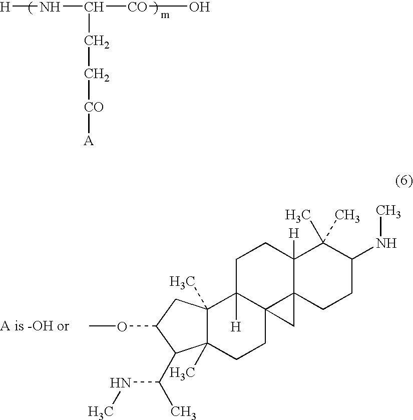

Preparation method was the same as that in Example 5, while α-methoxy-polyethylene-glycolω-glu-glu-glu was replaced with polyglutamic-acid (Mw 5000), product is N-(poly-glutamic-acid)-Cyclovirobuxine D (6). Yield: 90%.

### Example 7

### Synthesis of the Conjugate of Poly(Ethylene Glycol) Acetic Acid and Cyclovirobuxine D Having Ester Linkages Through a Amino Acid Linker

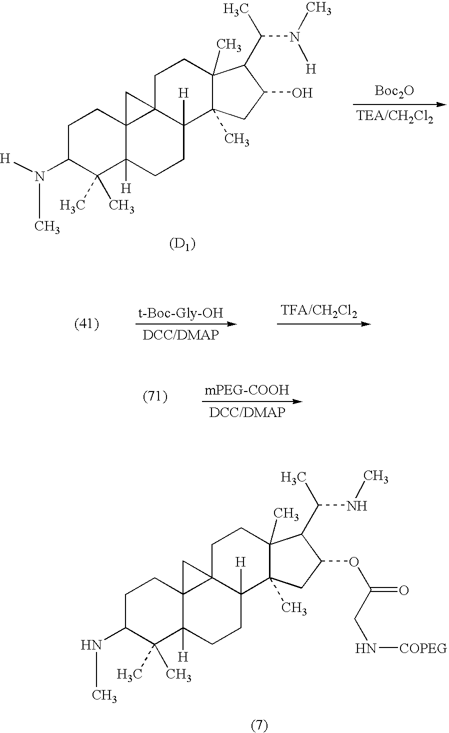

1.2 g tertiary-butyoxylcarboxylglycine (BOC-gly), 3.0 g N,N′-di(tertiary-butyoxylcarboxyl)-Cyclovirobuxine D (41) and 0.6 g 4-dimethylamine pyridine (DMAP) were dissolved in 30 ml of anhydrous dichloromethane. 1.45 g dicyclohexylcarbodiimide (DCC) was added thereafter. Reaction mixture was stirred over night at room temperature. Solid was filtered off and the organic layer was washed twice with sodium acetate (0.5M, pH 7), and then dried with anhydrous sodium sulfate, concentrated under reduced pressure. Then resulting concentrated semi-solid was dissolved in 8 ml of chloroform, and 6 ml trifluoroacetic acid was added dropwise in a period of 30 minutes. The solution was concentrated under reduced pressure. The obtained mixture was added into 20 ml of ether. The solution had a phase separation. The upper layer was removed. To the lower layer was added 30 ml of ethe. The mixture was thoroughly mixed and white precipitate was collected by filtration. The solid was washed by ether once, collected and dried under vacuum. Glycine-Cyclovirobuxine D ester (71). Yield: 3.8 g, NMR (DMSO): 0.40(s,1H), 0.51(s,1H), 4.96(t, 1H), 7.86(s, 1H), 8.03(s, 1H), 8.35(s,3H). Mp: 149-152° C.

0.5 g α-methoxy-polyethylene-glycol-ω-acetic-acid (Mw5000) and 110 mg glycine-Cyclovirobuxine D ester (71, obtained in the previous step, Mw 459), 25 mg 4-dimethylamine pyridine (DMAP) were dissolved in 10 ml of anhydrous dichloromethane and 2 ml of dimethylformate. 42 mg dicyclohexylcarbodiimide (DCC) were added thereafter. Reaction mixture was stirred over night at room temperature. The precipitate was filtered off, and the solution was concentrated under reduced pressure. The resulting mixture was added into 5 ml of isopropyl alcohol and 30 ml of ether. The product (α-methoxy-polyethylene-glycol-acetic-acid-gly-Cyclovirobuxine D ester (7)) was collected by filtration and dried under vacuum. Yield: 0.456 g. NMR (DMSO): 0.37(s,1H), 0.52(s,1H), 0.79(t, 6H), 3.5(br m, PEG-H).

### Example 8

### Synthesis of the Conjugate of Poly(Ethylene Glycol) Multicarboxyl Dipeptide and Cyclovirobuxine D Having Ester Linkages Through Amino Acid Linkers

1.0 g α-methoxy-polyethylene-glycol-ω-glu-glu-dipeptide (Mw10800) and 280 mg glycine-Cyclovirobuxine D ester (71), 50 mg 4-dimethylamine pyridine (DMAP) were dissolved in 15 ml anhydrous dichloromethane and 3 ml dimethylformate. 120 mg dicyclohexylcarbodiimide (DCC) were added thereafter. Reaction mixture was stirred over night at room temperature. The precipitate was filtered off, and the solution was concentrated under reduced pressure. The resulting mixture was added into 5 ml of isopropyl alcohol and 30 ml of ether. The product (α-methoxy-polyethylene-glycol-ω-glu-glu-gly-Cyclovirobuxine D ester (8)) was collected by filtration and dried under vacuum. Yield: 1.16 g, NMR (DMSO): 0.37(s,1H), 0.52(s,1H), 4.89(s, 2.6H), 3.5(br m, PEG-H).

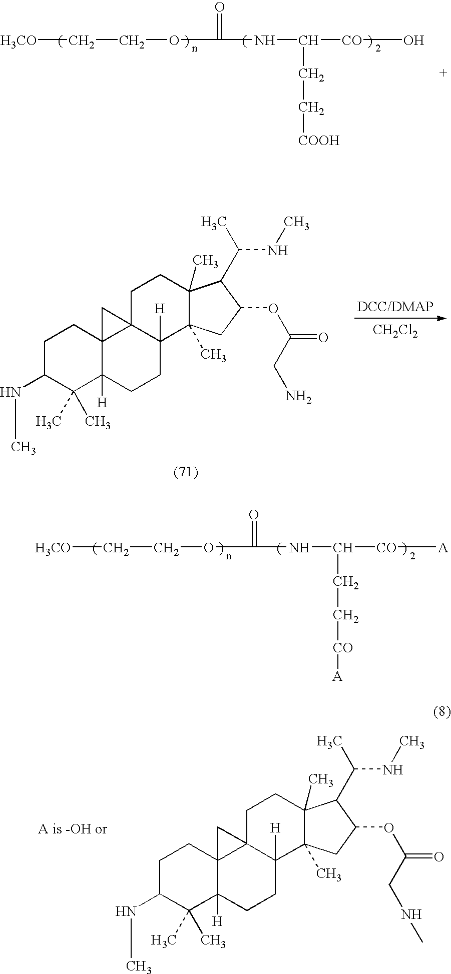

### Example 9

### Synthesis of the Conjugate of Poly(Ethylene Glycol) Multicarboxyl Tripeptide and Cyclovirobuxine D Having Ester Linkages

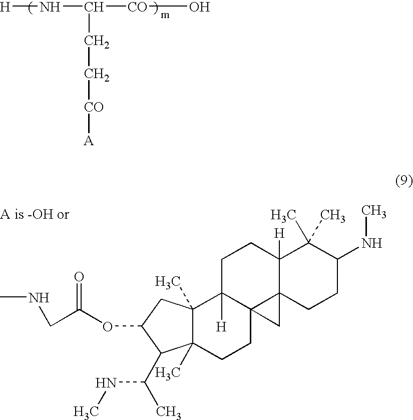

Preparation was the same as that in Example 8, while α-methoxy-polyethylene-glycol-ω-glu-glu was replaced with α-methoxy-polyethylene-glycol-ω-glu-glu-glu, and the product was α-methoxy-polyethylene-glycol-ω-glu-glu-glu-gly-Cyclovirobuxine D (9). Yield: 90%.

### Example 10

### Synthesis of the Conjugate of Poly-Glutamic-Acid and Cyclovirobuxine D Having Ester Linkages Through Amino Acid Linkers

Preparation was the same as that in Example 8, while α-methoxy-polyethylene-glycol-ω-glu-glu was replaced with poly-glutamic-acid, and product was poly-glutamic-acid-gly-Cyclovirobuxine D (10). Yield: 90%.

### Example 11

### Preparation of the Composition Containing the Conjugates

This example is to explain the preparation process of a typical composition administered non-gastrointestinally. The composition consists of the conjugate of the example 8.
Component Amount
Conjugate prepared in Example 82 g
0.9% saline to 100 ml

The conjugate prepared in example. 8 was dissolved in 0.9% saline to obtain 100 ml solution which was filtered through 0.2 μm membrane and packed aseptically for intravenous injection.

### Example 12

### Acute Toxicity and Therapeutic Efficacy Study of the Conjugates of PEG and Cyclovirobuxine D (Comparing with Cyclovirobuxine D)

The conjugate obtained from example 8 was used as the testing reagent in the following study, while native Cyclovirobuxine D was used as positive control. Both samples were dissolved in sodium acetate (pH 5.7) before injection.

I. Acute Toxicity Study

Kunming strain mice, 50% male and 50% formale, weighing 19-21 g, were randomly divided into 5 groups in the study. (1) native Cyclovirobuxine D, (2) the conjugate of PEG and Cyclovirobuxine D. The mice were fasted 12 hours before the injections. Each mice was given a single injection, and the volume for both IV and IP injection was based on 0.3 ml/10 g formulation. After injection, the mice were observed for 14 days, and their abnormal behavior and death were recorded. After 14 days, the mice still alive were executed and then analyzed.

Results:

1. LD₅₀ in IV injection: native Cyclovirobuxine D 11.77 mg/kg; the conjugate 35.50 mg/kg

2. LD₅₀ in IP injection: native Cyclovirobuxine D 97.52 mg/kg; the conjugate 144.98 mg/kg

II. The Effect on Pituitrin-Induced Myocardial Ischemic Injury in Mice

Methods: 50 normal SD rats of each sex, weighting 180-220 g, were randomly divided into five groups: controls, low dose of native Cyclovirobuxine D (0.25 mg/kg); middle dose of native Cyclovirobuxine D (0.5 mg/kg); low dose of the conjugate of Cyclovirobuxine D (0.25 mg/kg); middle dose of the conjugate of Cyclovirobuxine D (0.5 mg/kg). All rats were fasted before experiment for 12 hours, 1 g/kg urethane was administered by abdominal injection for anesthesia of the rats, and were placed in dorsal recumbency. A needle electrode was inserted into extremity subsurface carefully, examination leads ECG, holding 30 min, records the routine leads ECG The drug was given via vein of mice tail for each groups, after 10 min, 1.5 u/kg pituitrin was injected into dorsal veins of tongue, limited in 10 sec, records the routine leads ECG in 15 s, 30 s, 1, 2, 3, 5, 7, 10, 15 min after injected.

1. Effect on T Wave Change of Routine Leads ECG: table 1, table 2

2. Effect on cardioverter: table 3, table 4

TABLE 1
The effect of native Cyclovirobuxine D on the rising of T wave
by pituitrin-induced myocardial ischemic injury in mice (X ± S)

| | controls groups | middle dose groups | low dose groups |
|---|---|---|---|
| 0 | 0.18 ± 0.049 | 0.21 ± 0.052 | 0.20 ± 0.040 |
| 15″ | 0.54 ± 0.137 | 0.34 ± 0.119** | 0.50 ± 0.092 |
| 30″ | 0.29 ± 0.127 | 0.19 ± 0.116 | 0.23 ± 0.061 |
| 1′ | 0.25 ± 0.054 | 0.23 ± 0.068 | 0.22 ± 0.08 |
| 2′ | 0.23 ± 0.041 | 0.23 ± 0.051 | 0.18 ± 0.065 |
| 3′ | 0.24 ± 0.046 | 0.23 ± 0.047 | 0.21 ± 0.072 |
| 5′ | 0.25 ± 0.058 | 0.24 ± 0.040 | 0.22 ± 0.081 |
| 7′ | 0.23 ± 0.050 | 0.25 ± 0.048 | 0.21 ± 0.076 |
| 10′ | 0.23 ± 0.070 | 0.27 ± 0.044 | 0.23 ± 0.075 |
| 15′ | 0.24 ± 0.064 | 0.26 ± 0.040 | 0.24 ± 0.081 |

Comparison with the controls groups,
*P < 0.05,
**P < 0.01

TABLE 2
The effect of the conjugate of Cyclovirobuxine D on the rising of T
wave by pituitrin-induced myocardial ischemic injury in mice (X ± S)

| | controls groups | middle dose groups | low dose groups |
|---|---|---|---|
| 0 | 0.26 ± 0.067 | 0.23 ± 0.046 | 0.25 ± 0.064 |
| 15″ | 0.40 ± 0.088 | 0.33 ± 0.123 | 0.24 ± 0.059** |
| 30″ | 0.24 ± 0.16 | 0.22 ± 0.107 | 0.17 ± 0.123 |
| 1′ | 0.26 ± 0.131 | 0.28 ± 0.049 | 0.25 ± 0.062 |
| 2′ | 0.27 ± 0.076 | 0.27 ± 0.029 | 0.22 ± 0.102 |
| 3′ | 0.29 ± 0.071 | 0.28 ± 0.029 | 0.28 ± 0.071 |
| 5′ | 0.32 ± 0.059 | 0.31 ± 0.059 | 0.33 ± 0.056 |
| 7′ | 0.32 ± 0.055 | 0.31 ± 0.059 | 0.35 ± 0.067 |
| 10′ | 0.31 ± 0.059 | 0.30 ± 0.065 | 0.32 ± 0.052 |
| 15′ | 0.30 ± 0.049 | 0.33 ± 0.072 | 0.33 ± 0.069 |

Comparison with the controls groups,
*P < 0.05,
**P < 0.01

Observation: Native Cyclovirobuxine D (middle dose groups, 0.5 mg/kg) could inhibit the rising of T wave by pituitrin-induced in 15 sec, and comparing with the control group, the difference was significant (P<0.01); the conjugate of Cyclovirobuxine D (low dose groups, 0.25 mg/kg) could inhibit the rising of T wave by pituitrin-induced in 15 sec, and the difference was significant comparing with the control groups. (P<0.01)

TABLE 3
The effect of native Cyclovirobuxine D on the cardioverter by
pituitrin-induced myocardial ischemic injury in mice (X ± S)

| | controls groups | middle dose groups | low dose groups |
|---|---|---|---|
| 0 | 417.4 ± 29.35 | 389.6 ± 46.56 | 392.4 ± 48.11 |
| 15″ | 346.8 ± 60.98 | 331.1 ± 41.18 | 351.6 ± 90.78 |
| 30″ | 175 ± 62.25 | 234.5 ± 85.06 | 202.9 ± 42.85 |
| 1′ | 196.5 ± 60.55 | 196.2 ± 52.90 | 228.3 ± 44.41 |
| 2′ | 238.5 ± 53.76 | 215.4 ± 45.47 | 250.3 ± 29.14 |
| 3′ | 244.2 ± 59.53 | 236.7 ± 47.92 | 267.6 ± 44.71 |
| 5′ | 251.1 ± 46.99 | 256.6 ± 51.26 | 266.4 ± 30.09 |
| 7′ | 263 ± 47.24 | 268.6 ± 53.15 | 270.6 ± 30.52 |
| 10′ | 275.4 ± 51.80 | 260.8 ± 93.87 | 289.8 ± 43.91 |
| 15′ | 290.7 ± 40.04 | 297.6 ± 41.30 | 315.0 ± 63.39 |

TABLE 4
The effect of the conjugate of Cyclovirobuxine D on the cardioverter
by pituitrin-induced myocardial ischemic injury in mice (X ± S)

| | controls groups | middle dose groups | low dose groups |
|---|---|---|---|
| 0 | 400 ± 89.44 | 420 ± 60.00 | 420 ± 60.00 |
| 15″ | 320 ± 113.14 | 340 ± 44.72 | 270 ± 145.94 |
| 30″ | 200 ± 89.44 | 260 ± 107.70 | 200 ± 128.06 |
| 1′ | 190 ± 100.50 | 180 ± 60.00 | 150 ± 96.44 |
| 2′ | 240 ± 69.28 | 200 ± 56.57 | 180 ± 60.00 |
| 3′ | 200 ± 56.57 | 240 ± 0.00 | 200 ± 56.57 |
| 5′ | 220 ± 44.72 | 260 ± 44.72 | 240 ± 69.28 |
| 7′ | 240 ± 0.00 | 280 ± 56.57 | 260 ± 44.72 |
| 10′ | 260 ± 44.72 | 280 ± 56.57 | 260 ± 44.72 |
| 15′ | 300 ± 60.00 | 280 ± 56.57 | 300 ± 91.65 |

Observation: both native Cyclovirobuxine D and the conjugate of Cyclovirobuxine D have no significantly effect on the cardioverter by pituitrin-induced myocardial ischemic injury in mice, comparison with the controls groups. (P>0.05)

III. Result:

1. LD₅₀ (iv) and LD₅₀ (ip) of native Cyclovirobuxine D were 11.77 mg/kg and 97.52 mg/kg respectively. LD₅₀ (iv) and LD₅₀ (ip) of the conjugate of Cyclovirobuxine D were 35.50 mg/kg and 144.98 mg/kg correspondingly. LD₅₀ (iv) of the conjugate of Cyclovirobuxine D was significantly lower than that of the native Cyclovirobuxine D. In both iv and ip administration, LD₅₀ of the conjugate had lower value, meaning that the conjugate has lower toxicity than the native Cyclovirobuxine D.

2. Native Cyclovirobuxine D (middle dose groups, 0.5 mg/kg) could inhibit the rising of T wave by pituitrin-induced in 15 sec, and comparing with the control group, the difference was significant (P<0.01); the conjugate of Cyclovirobuxine D (low dose groups, 0.25 mg/kg) could inhibit the rising of T wave by pituitrin-induced in 15 sec, and the difference was significant comparing with the control groups. (P<0.01)

3. Native Cyclovirobuxine D and the conjugate of Cyclovirobuxine D had no significant effect on the cardioverter by pituitrin-induced myocardial ischemic injury in mice, comparing with the control groups. (P>0.05)

We claim:

1. A conjugate of hydrophilic polymers and the molecules from boxwood extraction or the synthetic derivatives of the molecules, represented by formula I:
(P-L [symbol] ₙD (I)
wherein:
P is a hydrophilic polymer, which is selected from a group consisting of polyethylene glycol, polyglutamic acid, polyaspartatic acid, polypropylene, polyvinyl alcohol, polyacrylmorpholine and their copolymer thereof;
n is an integer, not exceed the total number of hydroxy and amine group on the D;
L is a linking group, which is selected from a group consisting of: ester, carbonate, urethane, ether, acetal, and amide;
D is a molecule from the extraction of boxwood or its synthetic derivative, selected from a group consisting of Cyclovirobuxine D, Cycloprotobuxine A, Cycloprotobuxine C, Cyclovirobuxine C and their derivatives.

2. The conjugate of claim 1 wherein free hydroxyl on said hydrophilic polymer can be substituted by C₁₋₁₂ alkoxyl, cycloalkoxyl or aroxyl.

3. The conjugate of claim 1, wherein said, the hydrophilic polymer is polyethylene glycol, which has an average molecular weight from 300 to 60000.

4. A conjugate of claim 1, wherein said molecule from boxwood extraction is Cyclovirobuxine D, and the conjugate is represented by formula (I-D₁): 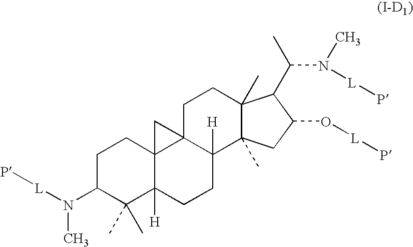
wherein:
P′ is independently selected from H or P, but not all be H at the same time;
L is a linker group as disclosed previously.

5. The conjugate of claim 1, wherein said, the linkage between the hydrophilic polymer and the molecule is an ester linker.

6. The conjugate of claim 1, wherein said, the conjugate is selected from a group consisting of N,N′-di(α-methoxy-ω-carboxyl-polyethylene glycol)-Cyclovirobuxine D (1), α-methoxy-polyethylene-glycol-acetic-acid-Cyclovirobuxine D ester (4), and α-methoxy-ω-carboxyl-polyethylene glycol-glycin-Cyclovirobuxine D ester (7).

7. A conjugate of the hydrophilic polymer containing multicarboxyl oligopeptide and the molecules from boxwood extraction or the synthetic derivatives of the molecules, represented by formula (II) 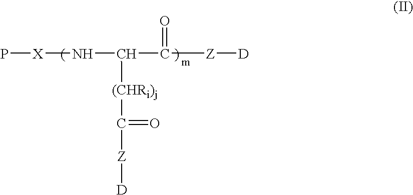
wherein:
P is a hydrophilic polymer, which is selected from a group consisting of polyethylene glycol, polyglutamic acid, polyaspartatic acid, polypropylene, polyvinyl alcohol, polyacrylmorpholine and their copolymer thereof;
m is an integer from 2 to 12;
j is an integer from 1 to 6;
Rᵢ is a group selected from H, C₁₋₁₂ alkyls, substituted aryls, aralkyls, heteroalkyls and substituted alkyls;
X is a linking group, selected from (CH₂)ₖ, (CH₂)ₖOCO, (CH₂)ₖNHCO or (CH₂)ₖCO, and k is 0-10;
Z is a linking group selected from O, NH, NHR, O(CH₂)ₕCOO or NH(CHR)ₕCOO, and h is 1-10;
D is the molecule from extraction of boxwood or its synthetic derivatives, preferring Cyclovirobuxine D.

8. The conjugate of claim 7, wherein said, the hydrophilic polymer is polyethylene glycol, which has an average molecular weight from 300 to 60000.

9. The conjugate of claim 7, wherein said, the conjugate is selected form a group consisting of N-(α-methoxy-polyethylene glycol-ω-glu-glu)-Cyclovirobuxine D (2); N-(poly-glutamic-acid)-Cyclovirobuxine D (3); α-methoxy-polyethylene glycol-ω-glu-glu-glu)-Cyclovirobuxine D ester (5); poly-glutamic-acid-Cyclovirobuxine D ester (6); α-methoxy-ω-carboxyl-polyethylene glycol-ω-glu-glu-glycin-Cyclovirobuxine D ester (8), and poly-glutamic-acid-glycin Cyclovirobuxine D ester (9).

10. A composition comprising any conjugate of claim 1-9 and pharmaceutically acceptable carrier and excipient.

11. The composition of claim 10, wherein said composition may be formulated into therapeutic acceptable formular, including tablet, suppository, pill, soft and hard gelatin capsules, powder, solution, suspension and aerosol.
